(19) United States
(12) Patent Application Publication
(10) Pub. No.: US 2008/0299215 A1
(43) Pub. Date: Dec. 4, 2008

(54) DEUTERIUM-ENRICHED LESTAURTINIB

(75) Inventor: Anthony W. Czarnik, Reno, NV (US)

Correspondence Address: VANCE INTELLECTUAL PROPERTY, PC, 5467 HILL TOP STREET, CROZET, VA, 22932-3167, US

(73) Assignee: PROTIA, LLC, Reno, NV (US)

(21) Appl. No.: 11/754,390

(22) Filed: May 29, 2007

(51) Int. Cl. A61K 33/00; A61P 13/10; C01B 4/00

(52) U.S. Cl. 424/600; 423/647.7

(57) ABSTRACT

The present application describes deuterium-enriched lestaurtinib, pharmaceutically acceptable salt forms thereof, and methods of treating using the same.

### FIELD OF THE INVENTION

This invention relates generally to deuterium-enriched lestaurtinib, pharmaceutical compositions containing the same, and methods of using the same.

### BACKGROUND OF THE INVENTION

Lestaurtinib, shown below, is a well known tyrosine kinase inhibitor.

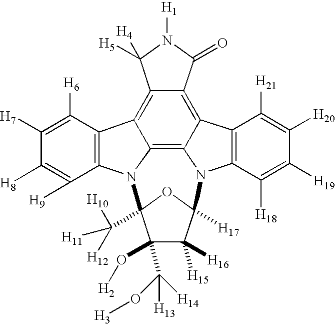

Since lestaurtinib is a known and useful pharmaceutical, it is desirable to discover novel derivatives thereof. Lestaurtinib is described in U.S. Pat. No. 4,923,986; the contents of which are incorporated herein by reference.

### SUMMARY OF THE INVENTION

Accordingly, one object of the present invention is to provide deuterium-enriched lestaurtinib or a pharmaceutically acceptable salt thereof.

It is another object of the present invention to provide pharmaceutical compositions comprising a pharmaceutically acceptable carrier and a therapeutically effective amount of at least one of the deuterium-enriched compounds of the present invention or a pharmaceutically acceptable salt thereof.

It is another object of the present invention to provide a method for treating acute myeloid leukemia, comprising administering to a host in need of such treatment a therapeutically effective amount of at least one of the deuterium-enriched compounds of the present invention or a pharmaceutically acceptable salt thereof.

It is another object of the present invention to provide a novel deuterium-enriched lestaurtinib or a pharmaceutically acceptable salt thereof for use in therapy.

It is another object of the present invention to provide the use of a novel deuterium-enriched lestaurtinib or a pharmaceutically acceptable salt thereof for the manufacture of a medicament (e.g., for the treatment of acute myeloid leukemia).

These and other objects, which will become apparent during the following detailed description, have been achieved by the inventor's discovery of the presently claimed deuterium-enriched lestaurtinib.

### DETAILED DESCRIPTION OF PREFERRED EMBODIMENTS

Deuterium (D or ²H) is a stable, non-radioactive isotope of hydrogen and has an atomic weight of 2.0144. Hydrogen naturally occurs as a mixture of the isotopes ¹H (hydrogen or protium), D (²H or deuterium), and T (³H or tritium). The natural abundance of deuterium is 0.015%. One of ordinary skill in the art recognizes that in all chemical compounds with a H atom, the H atom actually represents a mixture of H and D, with about 0.015% being D. Thus, compounds with a level of deuterium that has been enriched to be greater than its natural abundance of 0.015%, should be considered unnatural and, as a result, novel over their non-enriched counterparts.

All percentages given for the amount of deuterium present are mole percentages.

It can be quite difficult in the laboratory to achieve 100% deuteration at any one site of a lab scale amount of compound (e.g., milligram or greater). When 100% deuteration is recited or a deuterium atom is specifically shown in a structure, it is assumed that a small percentage of hydrogen may still be present. Deuterium-enriched can be achieved by either exchanging protons with deuterium or by synthesizing the molecule with enriched starting materials.

The present invention provides deuterium-enriched lestaurtinib or a pharmaceutically acceptable salt thereof. There are twenty-one hydrogen atoms in lestaurtinib as shown by variables R₁-R₂₁, in formula I below.

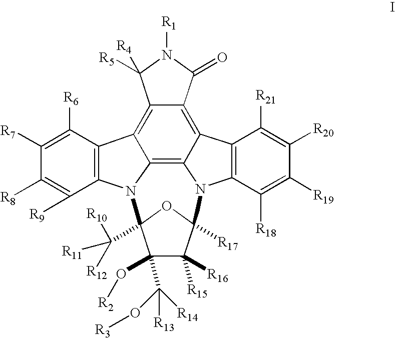

The hydrogens present on lestaurtinib have different capacities for exchange with deuterium. The hydrogen (or deuterium) atoms represented by R₁-R₃ are easily exchangeable with water under physiological conditions. Thus, if any of R₁-R₃ are deuterium, they will readily exchange with a proton after administration to a patient. The hydrogens represented by R₄-R₅ should be exchangeable with deuterium in the presence of deuterated base (e.g., KOt-Bu in DOt-Bu).

The present invention is based on increasing the amount of deuterium present in lestaurtinib above its natural abundance. This increasing is called enrichment or deuterium-enrichment. If not specifically noted, the percentage of enrichment refers to the percentage of deuterium present in the compound, mixture of compounds, or composition. Examples of the amount of enrichment include from about 0.5, 1, 2, 3, 4, 5, 6, 7, 8, 9, 10, 12, 16, 21, 25, 29, 33, 37, 42, 46, 50, 54, 58, 63, 67, 71, 75, 79, 84, 88, 92, 96, to about 100 mol %. Since there are 21 hydrogens in lestaurtinib, replacement of a single hydrogen atom with deuterium would result in a molecule with about 5% deuterium enrichment. In order to achieve enrichment less than about 5%, but above the natural abundance, only partial deuteration of one site is required. Thus, less than about 5% enrichment would still refer to deuterium-enriched lestaurtinib.

With the natural abundance of deuterium being 0.015%, one would expect that for approximately every 6,667 molecules of lestaurtinib (1/0.00015=6,667), there is one naturally occurring molecule with one deuterium present. Since lestaurtinib has 21 positions, one would roughly expect that for approximately every 140,007 molecules of lestaurtinib (21×6,667), all 21 different, naturally occurring, mono-deuterated lestaurtinibs would be present. This approximation is a rough estimate as it doesn't take into account the different exchange rates of the hydrogen atoms on lestaurtinib. For naturally occurring molecules with more than one deuterium, the numbers become vastly larger. In view of this natural abundance, the present invention, in an embodiment, relates to an amount of a deuterium enriched compound, whereby the enrichment recited will be more than naturally occurring deuterated molecules.

In view of the natural abundance of deuterium-enriched lestaurtinib, the present invention also relates to isolated or purified deuterium-enriched lestaurtinib. The isolated or purified deuterium-enriched lestaurtinib is a group of molecules whose deuterium levels are above the naturally occurring levels (e.g., 5%). The isolated or purified deuterium-enriched lestaurtinib can be obtained by techniques known to those of skill in the art (e.g., see the syntheses described below).

The present invention also relates to compositions comprising deuterium-enriched lestaurtinib. The compositions require the presence of deuterium-enriched lestaurtinib which is greater than its natural abundance. For example, the compositions of the present invention can comprise (a) a μg of a deuterium-enriched lestaurtinib; (b) a mg of a deuterium-enriched lestaurtinib; and, (c) a gram of a deuterium-enriched lestaurtinib.

In an embodiment, the present invention provides an amount of a novel deuterium-enriched lestaurtinib.

Examples of amounts include, but are not limited to (a) at least 0.01, 0.02, 0.03, 0.04, 0.05, 0.1, 0.2, 0.3, 0.4, 0.5, to 1 mole, (b) at least 0.1 moles, and (c) at least 1 mole of the compound. The present amounts also cover lab-scale (e.g., gram scale), kilo-lab scale (e.g., kilogram scale), and industrial or commercial scale (e.g., multi-kilogram or above scale) quantities as these will be more useful in the actual manufacture of a pharmaceutical. Industrial/commercial scale refers to the amount of product that would be produced in a batch that was designed for clinical testing, formulation, sale/distribution to the public, etc.

In another embodiment, the present invention provides a novel, deuterium enriched compound of formula I or a pharmaceutically acceptable salt thereof.

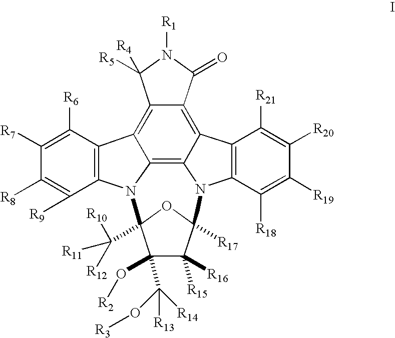

wherein R₁-R₂₁, are independently selected from H and D; and the abundance of deuterium in R₁-R₂₁ is at least 5%. The abundance can also be (a) at least 10%, (b) at least 14%, (c) at least 19%, (d) at least 24%, (e) at least 29%, (f) at least 33%, (g) at least 38%, (h) at least 43%, (i) at least 48%, (j) at least 52%, (k) at least 57%, (l) at least 62%, (m) at least 67%, (n) at least 71%, (O) at least 76%, (p) at least 81%, (q) at least 86%, (r) at least 90%, (s) at least 95%, and (t) 100%.

In another embodiment, the present invention provides a novel, deuterium enriched compound of formula I or a pharmaceutically acceptable salt thereof, wherein the abundance of deuterium in R₁-R₃ is at least 33%. The abundance can also be (a) at least 67%, and (b) 100%.

In another embodiment, the present invention provides a novel, deuterium enriched compound of formula I or a pharmaceutically acceptable salt thereof, wherein the abundance of deuterium in R₄-R₅ is at least 50%. The abundance can also be 100%.

In another embodiment, the present invention provides a novel, deuterium enriched compound of formula I or a pharmaceutically acceptable salt thereof, wherein the abundance of deuterium in R₆-R₉ and R₁₈-R₂₁ is at least 13%. The abundance can also be (a) at least 25%, (b) at least 38%, (c) at least 50%, (d) at least 63%, (e) at least 75%, (f) at least 88%, and (g) 100%.

In another embodiment, the present invention provides a novel, deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides an isolated novel, deuterium enriched compound of formula I or a pharmaceutically acceptable salt thereof.

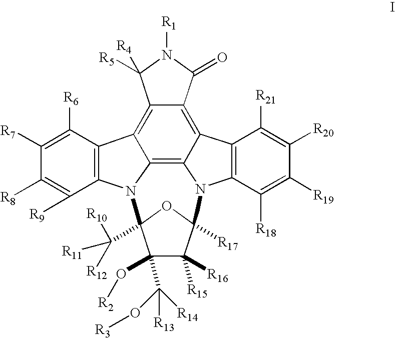

wherein R₁-R₂₁, are independently selected from H and D; and the abundance of deuterium in R₁-R₂₁ is at least 5%. The abundance can also be (a) at least 10%, (b) at least 14%, (c) at least 19%, (d) at least 24%, (e) at least 29%, (f) at least 33%, (g) at least 38%, (h) at least 43%, (i) at least 48%, (j) at least 52%, (k) at least 57%, (l) at least 62%, (m) at least 67%, (n) at least 71%, (O) at least 76%, (p) at least 81%, (q) at least 86%, (r) at least 90%, (s) at least 95%, and (t) 100%.

In another embodiment, the present invention provides an isolated novel, deuterium enriched compound of formula I or a pharmaceutically acceptable salt thereof, wherein the abundance of deuterium in R₁-R₃ is at least 33%. The abundance can also be (a) at least 67%, and (b) 100%.

In another embodiment, the present invention provides an isolated novel, deuterium enriched compound of formula I or a pharmaceutically acceptable salt thereof, wherein the abundance of deuterium in R₄-R₅ is at least 50%. The abundance can also be 100%.

In another embodiment, the present invention provides an isolated novel, deuterium enriched compound of formula I, wherein the abundance of deuterium in R₁-R₃ and R₄-R₅ is at least 20%. The abundance can also be (a) at least 40%, (b) at least 60%, (c) at least 80%, and (d) 100%.

In another embodiment, the present invention provides an isolated novel, deuterium enriched compound of formula I or a pharmaceutically acceptable salt thereof, wherein the abundance of deuterium in R₆-R₉ and R₁₈-R₂₁ is at least 13%. The abundance can also be (a) at least 25%, (b) at least 38%, (c) at least 50%, (d) at least 63%, (e) at least 75%, (f) at least 88%, and (g) 100%.

In another embodiment, the present invention provides an isolated novel, deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides novel mixture of deuterium enriched compounds of formula I or a pharmaceutically acceptable salt thereof.

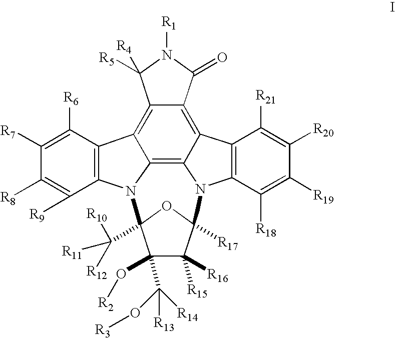

wherein R₁-R₂₁, are independently selected from H and D; and the abundance of deuterium in R₁-R₂₁ is at least 5%. The abundance can also be (a) at least 10%, (b) at least 14%, (c) at least 19%, (d) at least 24%, (e) at least 29%, (f) at least 33%, (g) at least 38%, (h) at least 43%, (i) at least 48%, (j) at least 52%, (k) at least 57%, (l) at least 62%, (m) at least 67%, (n) at least 71%, (O) at least 76%, (p) at least 81%, (q) at least 86%, (r) at least 90%, (s) at least 95%, and (t) 100%.

In another embodiment, the present invention provides a novel mixture of, deuterium enriched compound of formula I or a pharmaceutically acceptable salt thereof, wherein the abundance of deuterium in R₁-R₃ is at least 33%. The abundance can also be (a) at least 67%, and (b) 100%.

In another embodiment, the present invention provides a novel mixture of, deuterium enriched compound of formula I or a pharmaceutically acceptable salt thereof, wherein the abundance of deuterium in R₄-R₅ is at least 50%. The abundance can also be 100%.

In another embodiment, the present invention provides a novel mixture of, deuterium enriched compound of formula I, wherein the abundance of deuterium in R₁-R₃ and R₄-R₅ is at least 20%. The abundance can also be (a) at least 40%, (b) at least 60%, (c) at least 80%, and (d) 100%.

In another embodiment, the present invention provides a novel mixture of, deuterium enriched compound of formula I or a pharmaceutically acceptable salt thereof, wherein the abundance of deuterium in R₆-R₉ and R₁₈-R₂₁ is at least 13%. The abundance can also be (a) at least 25%, (b) at least 38%, (c) at least 50%, (d) at least 63%, (e) at least 75%, (f) at least 88%, and (g) 100%.

In another embodiment, the present invention provides a novel mixture of, deuterium enriched compound of formula I or a pharmaceutically acceptable salt thereof, wherein the abundance of deuterium in R₁₀-R₁₂ is at least 33%. The abundance can also be (a) at least 67%, and (b) 100%.

In another embodiment, the present invention provides novel pharmaceutical compositions, comprising: a pharmaceutically acceptable carrier and a therapeutically effective amount of a deuterium-enriched compound of the present invention.

In another embodiment, the present invention provides a novel method for treating acute myeloid leukemia comprising: administering to a patient in need thereof a therapeutically effective amount of a deuterium-enriched compound of the present invention.

In another embodiment, the present invention provides an amount of a deuterium-enriched compound of the present invention as described above for use in therapy.

In another embodiment, the present invention provides the use of an amount of a deuterium-enriched compound of the present invention for the manufacture of a medicament (e.g., for the treatment of acute myeloid leukemia).

The present invention may be embodied in other specific forms without departing from the spirit or essential attributes thereof. This invention encompasses all combinations of preferred aspects of the invention noted herein. It is understood that any and all embodiments of the present invention may be taken in conjunction with any other embodiment or embodiments to describe additional more preferred embodiments. It is also to be understood that each individual element of the preferred embodiments is intended to be taken individually as its own independent preferred embodiment. Furthermore, any element of an embodiment is meant to be combined with any and all other elements from any embodiment to describe an additional embodiment.

### Definitions

The examples provided in the definitions present in this application are non-inclusive unless otherwise stated. They include but are not limited to the recited examples.

The compounds of the present invention may have asymmetric centers. Compounds of the present invention containing an asymmetrically substituted atom may be isolated in optically active or racemic forms. It is well known in the art how to prepare optically active forms, such as by resolution of racemic forms or by synthesis from optically active starting materials. All processes used to prepare compounds of the present invention and intermediates made therein are considered to be part of the present invention. All tautomers of shown or described compounds are also considered to be part of the present invention.

“Host” or “patient” typically refers to a human. It also includes other mammals including the equine, porcine, bovine, feline, and canine families.

“Treating” or “treatment” covers the treatment of a disease-state in a mammal, and includes: (a) preventing the disease-state from occurring in a mammal, in particular, when such mammal is predisposed to the disease-state but has not yet been diagnosed as having it; (b) inhibiting the disease-state, e.g., arresting it development; and/or (c) relieving the disease-state, e.g., causing regression of the disease state until a desired endpoint is reached. Treating also includes the amelioration of a symptom of a disease (e.g., lessen the pain or discomfort), wherein such amelioration may or may not be directly affecting the disease (e.g., cause, transmission, expression, etc.).

“Therapeutically effective amount” includes an amount of a compound of the present invention that is effective when administered alone or in combination to treat the desired condition or disorder. “Therapeutically effective amount” includes an amount of the combination of compounds claimed that is effective to treat the desired condition or disorder. The combination of compounds is preferably a synergistic combination. Synergy, as described, for example, by Chou and Talalay, Adv. Enzyme Regul. 1984, 22:27-55, occurs when the effect of the compounds when administered in combination is greater than the additive effect of the compounds when administered alone as a single agent. In general, a synergistic effect is most clearly demonstrated at sub-optimal concentrations of the compounds. Synergy can be in terms of lower cytotoxicity, increased antiviral effect, or some other beneficial effect of the combination compared with the individual components.

“Pharmaceutically acceptable salts” refer to derivatives of the disclosed compounds wherein the parent compound is modified by making acid or base salts thereof. Examples of pharmaceutically acceptable salts include, but are not limited to, mineral or organic acid salts of the basic residues. The pharmaceutically acceptable salts include the conventional quaternary ammonium salts of the parent compound formed, for example, from non-toxic inorganic or organic acids. For example, such conventional non-toxic salts include, but are not limited to, those derived from inorganic and organic acids selected from 1, 2-ethanedisulfonic, 2-acetoxybenzoic, 2-hydroxyethanesulfonic, acetic, ascorbic, benzenesulfonic, benzoic, bicarbonic, carbonic, citric, edetic, ethane disulfonic, ethane sulfonic, fumaric, glucoheptonic, gluconic, glutamic, glycolic, glycollyarsanilic, hexylresorcinic, hydrabamic, hydrobromic, hydrochloric, hydroiodide, hydroxymaleic, hydroxynaphthoic, isethionic, lactic, lactobionic, lauryl sulfonic, maleic, malic, mandelic, methanesulfonic, napsylic, nitric, oxalic, pamoic, pantothenic, phenylacetic, phosphoric, polygalacturonic, propionic, salicyclic, stearic, subacetic, succinic, sulfamic, sulfanilic, sulfuric, tannic, tartaric, and toluenesulfonic.

### Synthesis

Scheme 1 shows two examples of how to prepare lestaurtinib. In the first example, (+)-K-252a (5), isolated from Streptomyces staurosporeus, is reduced by lithium aluminum hydride or lithium triethylborohydride to produce lestaurtinib. See for example Murakata, et al., U.S. Pat. No. 4,923,986 and Gingrich, et al., J. Med. Chem. 2005, 48, 3776-3783. In the second example, 5 is made synthetically rather than isolated from nature. A multi-step synthesis involving key components 1, 3, and 3 is employed to make the ketone 4, which may be used to make 5 and thus lestaurtinib. See Fukuyama, et al., Jpn. Kokai Tokkyo Koho, 2000247976 and J. Am. Chem. Soc., 1999, 121, 6501-6502. A key feature of this synthesis is that the two indole moieties are introduced in a stepwise fashion, allowing the possibility that deuterium atoms may be placed on one or both of the indole benzene rings in a controlled fashion.

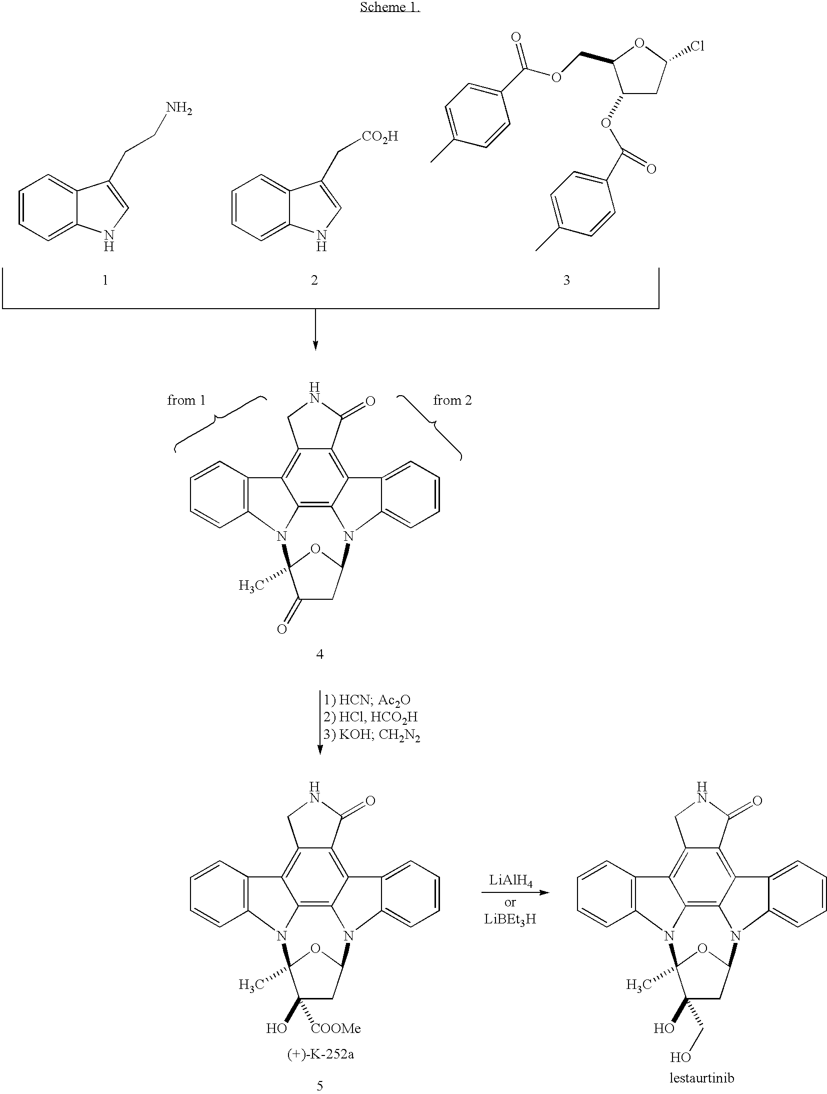

Scheme 2 shows how lestaurtinib can be formed. Equation (l), the use of lithium aluminum deuteride or lithium triethylborodeuteride in the reduction of 5 produces 6, which is lestaurtinib with R₁₃-R₁₄=D. Electrophilic bromination of lestaurtinib followed by deuterolysis of the resultant dibromide as shown in equation (2) will afford 7, which is lestaurtinib with R₇ and R₂₀=D. The use of the known (Raisanen, et al., Acta Chem. Scand. B, 1979, B33, 11-14) pentadeuterated tryptamine 8 in the chemistry of Scheme 1 will produce 9, which is lestaurtinib with R₆-R₉=D. See equation (3) of Scheme 2. The use of commercially available pentadeuterioindole-3-acetic acid 10 in the chemistry of Scheme 1 will produce 11, which is lestaurtinib with R₁₈-R₂₁, =D. See equation (4) of Scheme 2. The use of various known monodeuterated versions of tryptamine and indole-3-acetic acid, namely 12-15, will lead to other deuterated forms of lestaurtinib when used in the chemistry of Scheme 1. Specifically, 12 will produce lestaurtinib with R₆=D, 13 will produce lestaurtinib with R₉=D, 14 will produce lestaurtinib with R₂₁=D, and 15 will produce lestaurtinib with R₂₀=D.

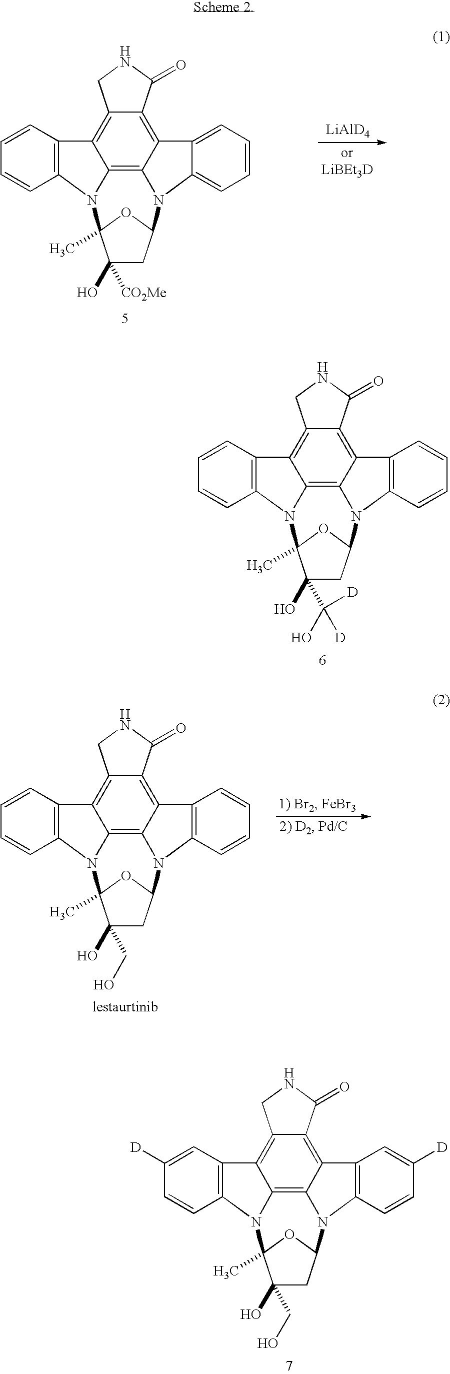

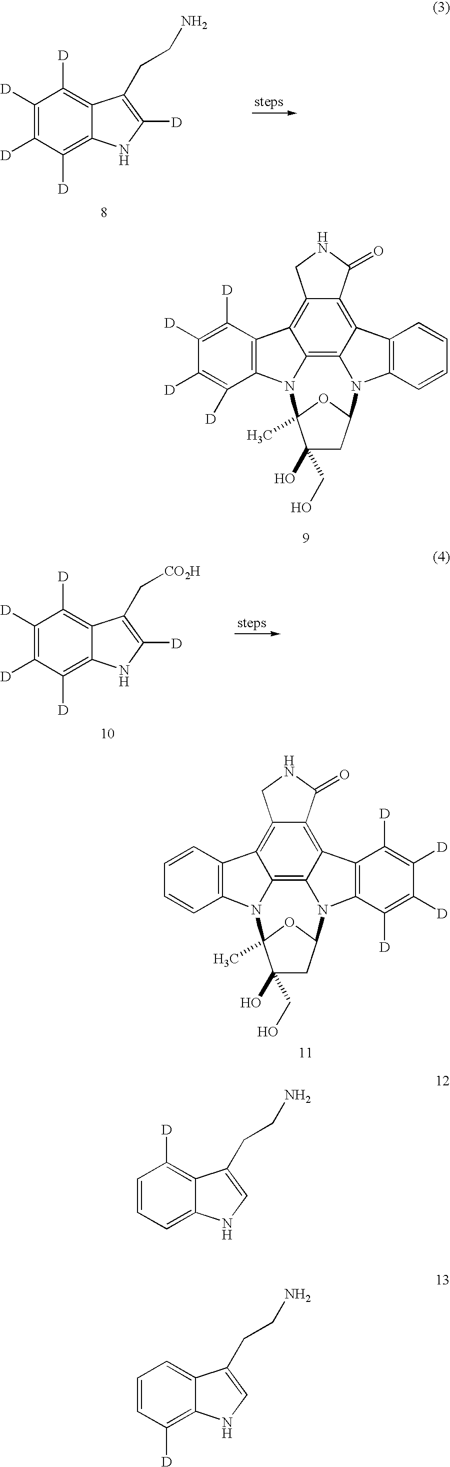

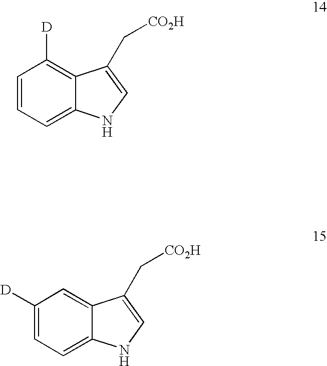

Scheme 3 shows the ketone 4 may be obtained synthetically as shown in Scheme 1 or from lestaurtinib itself by oxidation as shown in Scheme 3 (Gingrich, et al., J. Med. Chem. 2005, 48, 3776-3783). Exchange of the two hydrogen atoms next to the ketone may then be accomplished, affording 16. Introduction of the carbomethoxy group as shown (see also Scheme 1) affords 17, which after reduction produces the deuterated lestaurtinib analogs 18 (lestaurtinib with R₁₅-R₁₆=D) and 19 (lestaurtinib with R₁₃-R₁₆=D).

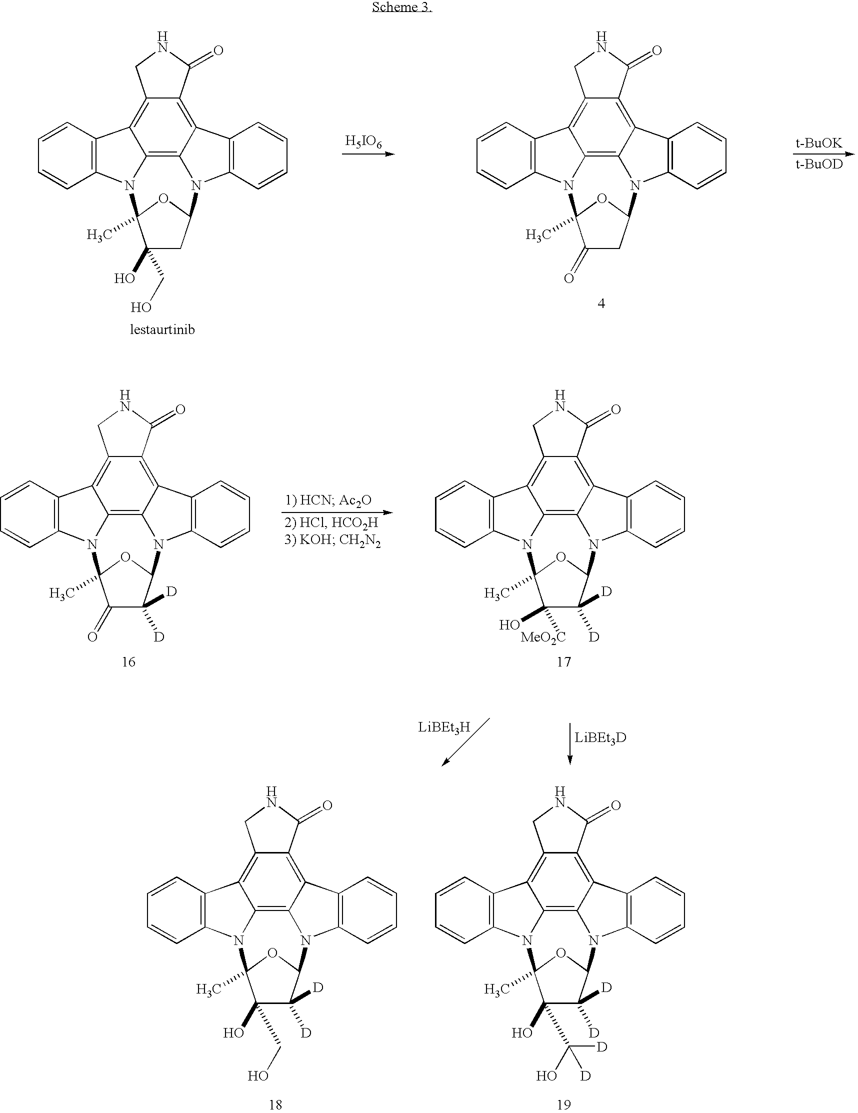

Using combinations of the various deuterated starting materials and intermediates shown in Schemes 4 and 5, a person skilled in the art of organic chemistry should be able to prepare a wide variety of deuterated lestaurtinib analogs.

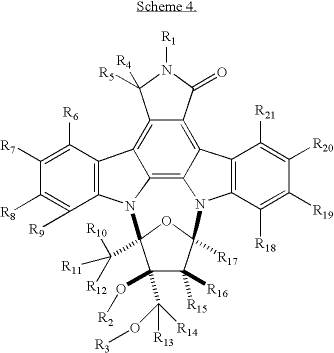

Using combinations of the various deuterated starting materials and intermediates shown in Schemes 4 and 5, a person skilled in the art of organic chemistry should be able to prepare a wide variety of deuterated lestaurtinib analogs.

### EXAMPLES

| | |
|---|---|
| 1 | 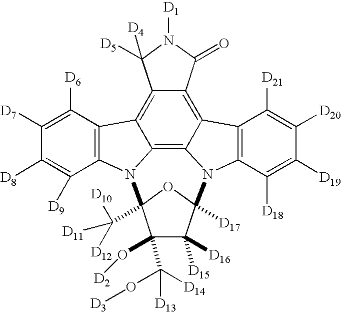 |
| 2 | 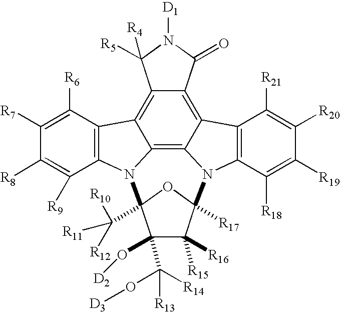 |
| 3 | 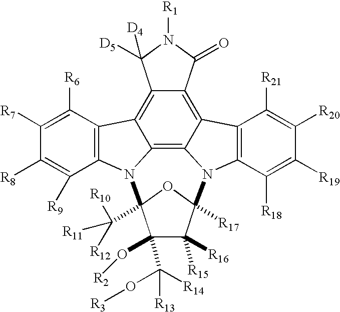 |
| 4 | 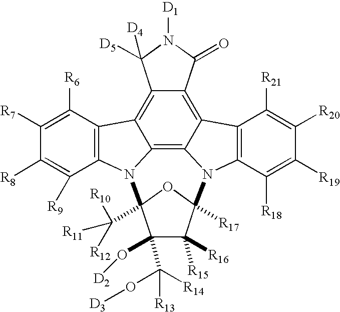 |
| 5 | 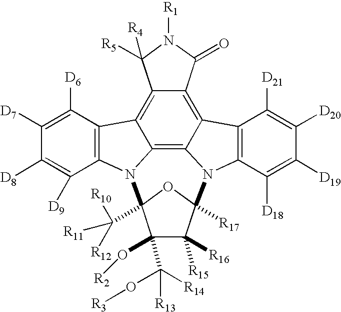 |
| 6 | 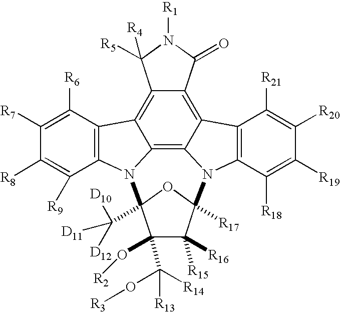 |
| 7 | 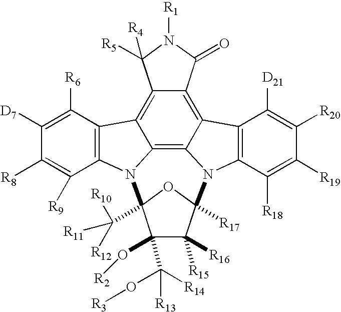 |
| 8 | 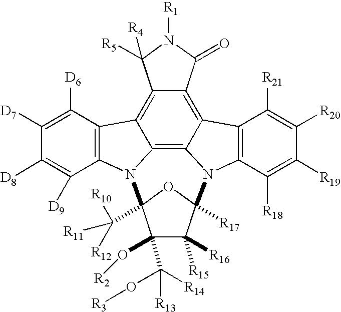 |
| 9 | 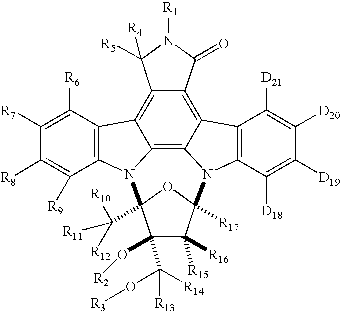 |
| 10 | 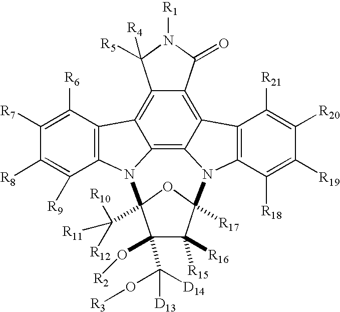 |
| 11 | 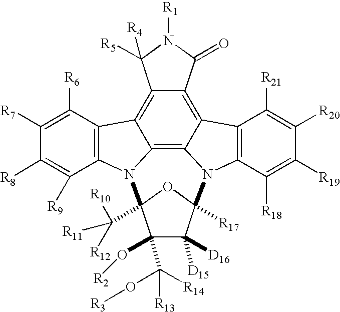 |
| 12 | 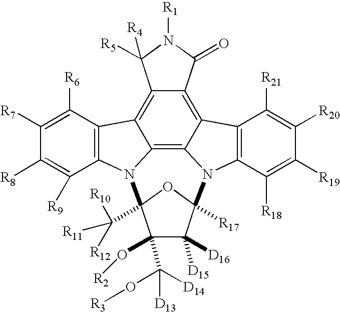 |

Table 2 provides compounds that are representative examples of the present invention. Where H is shown, it represents naturally abundant hydrogen.

| | |
|---|---|
| 13 | 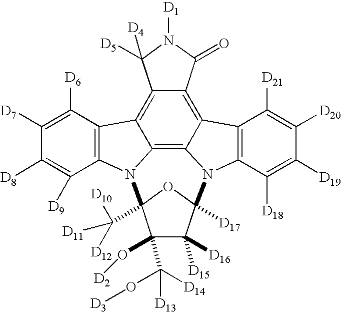 |
| 14 | 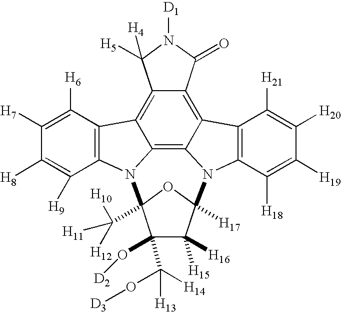 |
| 15 | 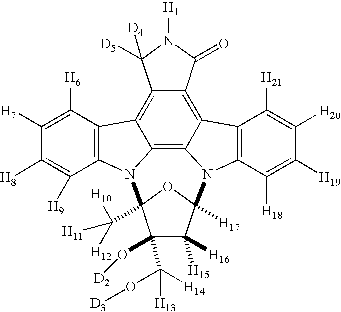 |
| 16 | 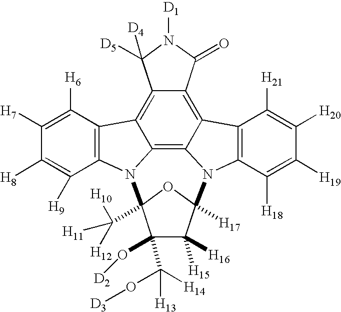 |
| 17 | 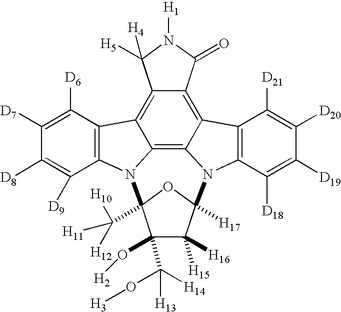 |
| 18 | 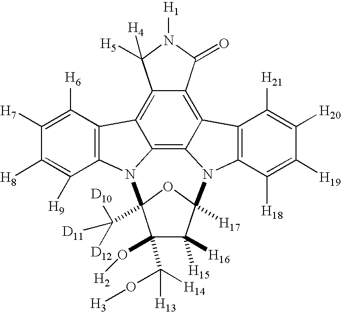 |
| 19 | 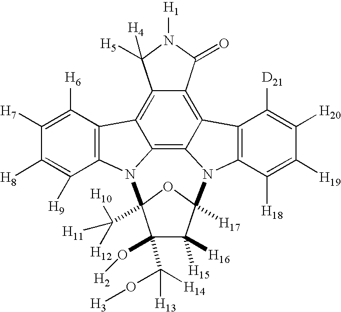 |
| 20 | 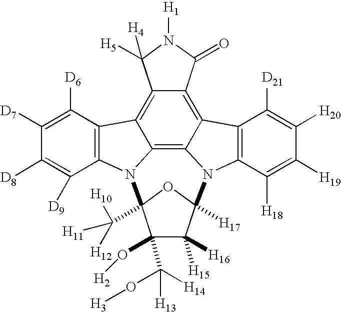 |
| 21 | 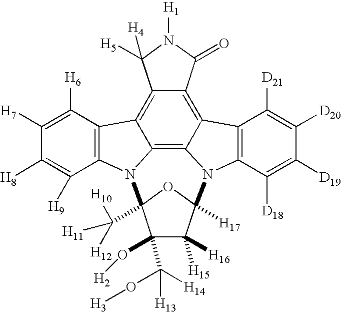 |
| 22 | 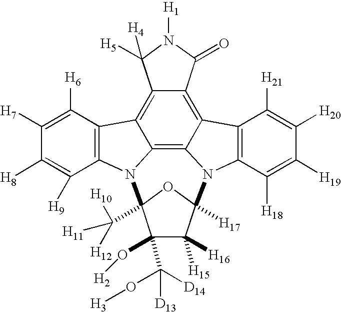 |
| 23 | 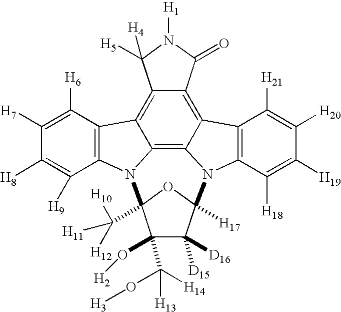 |
| 24 | 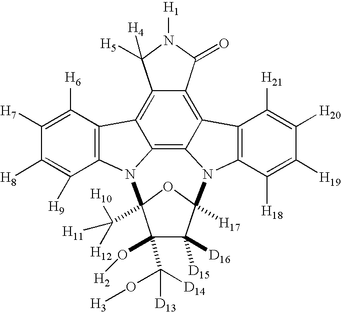 |

Numerous modifications and variations of the present invention are possible in light of the above teachings. It is therefore to be understood that within the scope of the appended claims, the invention may be practiced otherwise that as specifically described herein.

What is claimed is:

1. A deuterium-enriched compound of formula I or a pharmaceutically acceptable salt thereof: 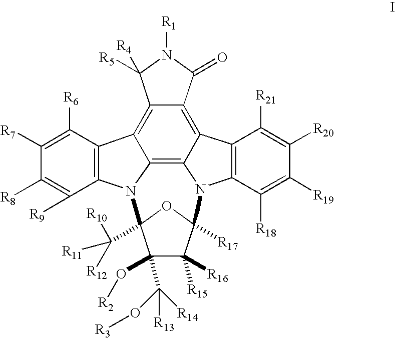
wherein R₁-R₂₁, are independently selected from H and D; and
the abundance of deuterium in R₁-R₂₁ is at least 5%.

2. A deuterium-enriched compound of claim 1, wherein the abundance of deuterium in R₁-R₂₁, is selected from at least 5%, at least 10%, at least 14%, at least 19%, at least 24%, at least 29%, at least 33%, at least 38%, at least 43%, at least 48%, at least 52%, (k) at least 57%, at least 62%, at least 67%, at least 71%, at least 76%, at least 81%, at least 86%, at least 90%, at least 95%, and 100%.

3. A deuterium-enriched compound of claim 1, wherein the abundance of deuterium in R₁-R₃ is selected from at least 33%, at least 67%, and 100%.

4. A deuterium-enriched compound of claim 1, wherein the abundance of deuterium in R₄-R₅ is selected from at least 50% and 100%.

5. A deuterium-enriched compound of claim 1, wherein the abundance of deuterium in R₁-R₃ and R₄-R₅ is selected from at least 20%, at least 40%, at least 60%, at least 80%, and 100%.

6. A deuterium-enriched compound of claim 1, wherein the abundance of deuterium in R₆-R₉ and R₁₈-R₂₁ is selected from at least 13%, at least 25%, at least 38%, at least 50%, at least 63%, at least 75%, at least 88%, and 100%.

7. A deuterium-enriched compound of claim 1, wherein the abundance of deuterium in R₁₀-R₁₂ is selected from at least 33%, at least 67%, and 100%.

8. A deuterium-enriched compound of claim 1, wherein the compound is selected from compounds 1-12 of Table 1:

9. A deuterium-enriched compound of claim 1, wherein the compound is selected from compounds 13-24 of Table 2:

10. An isolated deuterium-enriched compound of formula I or a pharmaceutically acceptable salt thereof: 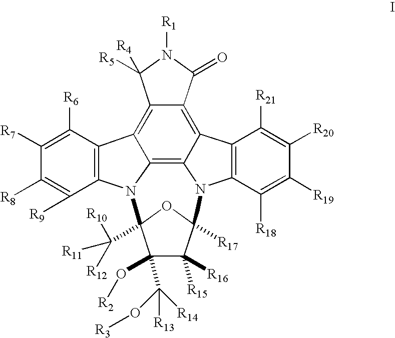
wherein R₁-R₂₁, are independently selected from H and D; and
the abundance of deuterium in R₁-R₂₁ is at least 5%.

11. An isolated deuterium-enriched compound of claim 10, wherein the abundance of deuterium in R₁-R₂₁ is selected from at least 5%, at least 10%, at least 14%, at least 19%, at least 24%, at least 29%, at least 33%, at least 38%, at least 43%, at least 48%, at least 52%, (k) at least 57%, at least 62%, at least 67%, at least 71%, at least 76%, at least 81%, at least 86%, at least 90%, at least 95%, and 100%.

12. An isolated deuterium-enriched compound of claim 10, wherein the abundance of deuterium in R₁-R₃ is selected from at least 33%, at least 67%, and 100%.

13. An isolated deuterium-enriched compound of claim 10, wherein the abundance of deuterium in R₄-R₅ is selected from at least 50% and 100%.

14. An isolated deuterium-enriched compound of claim 10, wherein the abundance of deuterium in R₁-R₃ and R₄-R₅ is selected from at least 20%, at least 40%, at least 60%, at least 80%, and 100%.

15. An isolated deuterium-enriched compound of claim 10, wherein the abundance of deuterium in R₆-R₉ and R₁₈-R₂₁, is selected from at least 13%, at least 25%, at least 38%, at least 50%, at least 63%, at least 75%, at least 88%, and 100%.

16. An isolated deuterium-enriched compound of claim 10, wherein the abundance of deuterium in R₁₀-R₁₂ is selected from at least 33%, at least 67%, and 100%.

17. An isolated deuterium-enriched compound of claim 10, wherein the compound is selected from compounds 1-12 of Table 1:

18. An isolated deuterium-enriched compound of claim 10, wherein the compound is selected from compounds 13-24 of Table 2:

19. A mixture of deuterium-enriched compounds of formula I or a pharmaceutically acceptable salt thereof: 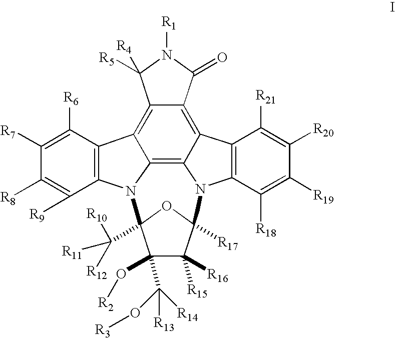
wherein R₁-R₂₁, are independently selected from H and D; and
the abundance of deuterium in R₁-R₂₁ is at least 5%.

20. A mixture of deuterium-enriched compound of claim 19, wherein the abundance of deuterium in R₁-R₂₁ is selected from at least 5%, at least 10%, at least 14%, at least 19%, at least 24%, at least 29%, at least 33%, at least 38%, at least 43%, at least 48%, at least 52%, (k) at least 57%, at least 62%, at least 67%, at least 71%, at least 76%, at least 81%, at least 86%, at least 90%, at least 95%, and 100%.

21. A mixture of deuterium-enriched compound of claim 19, wherein the abundance of deuterium in R₁-R₃ is selected from at least 33%, at least 67%, and 100%.

22. A mixture of deuterium-enriched compound of claim 19, wherein the abundance of deuterium in R₄-R₅ is selected from at least 50% and 100%.

23. A mixture of deuterium-enriched compound of claim 19, wherein the abundance of deuterium in R₁-R₃ and R₄-R₅ is selected from at least 20%, at least 40%, at least 60%, at least 80%, and 100%.

24. A mixture of deuterium-enriched compound of claim 19, wherein the abundance of deuterium in R₆-R₉ and R₁₈-R₂₁, is selected from at least 13%, at least 25%, at least 38%, at least 50%, at least 63%, at least 75%, at least 88%, and 100%.

25. A mixture of deuterium-enriched compound of claim 19, wherein the abundance of deuterium in R₁₀-R₁₂ is selected from at least 33%, at least 67%, and 100%.

26. A mixture of deuterium-enriched compounds of claim 19, wherein the compounds are selected from compounds 1-12 of Table 1:

27. A mixture of deuterium-enriched compounds of claim 19, wherein the compounds are selected from compounds 13-24 of Table 2:

28. A pharmaceutical composition, comprising: a pharmaceutically acceptable carrier and a therapeutically effective amount of a compound of claim 1-27 or a pharmaceutically acceptable salt form thereof.

29. A method for treating acute myeloid leukemia comprising: administering, to a patient in need thereof, a therapeutically effective amount of a compound of claim 1-27 or a pharmaceutically acceptable salt form thereof.
